(19) United States
(12) Patent Application Publication
(10) Pub. No.: US 2015/0290236 A1
(43) Pub. Date: Oct. 15, 2015

(54) PHARMACEUTICAL COMPOSITIONS OF REGADENOSON

(71) Applicant: LEIUTIS PHARMACEUTICALS PVT. LTD., Andhra Pradesh (IN)

(72) Inventors: Kocherlakota CHANDRASHEKHAR, Secunderabad (IN); Banda NAGARAJU, Hyderabad (IN)

(21) Appl. No.: 14/648,525

(22) PCT Filed: Nov. 27, 2013
(86) PCT No.: PCT/IN2013/000719

§ 371 Date: May 29, 2015

(30) Foreign Application Priority Data
Nov. 30, 2012 (IN) 4997/CHE/2012

(51) Int. Cl. A61K 31/7076; A61K 47/10; A61K 47/02

(52) U.S. Cl. CPC A61K 31/7076; A61K 47/02; A61K 47/10

(57) ABSTRACT

The present invention relates to novel pharmaceutical compositions of Regadenoson and its pharmaceutically acceptable salts, solvates or hydrates in the form of solution, wherein the compositions are free from phosphate buffer and EDTA. Further the invention relates to pharmaceutical composition of Regadenoson comprising a tonidty modifier.

### FIELD OF INVENTION

The present invention relates to the pharmaceutical composition of Regadenoson including its pharmaceutically acceptable salts, solvates, hydrates and polymorphs thereof, devoid of phosphate buffer and chelating agents. Further the invention relates to pharmaceutical composition of Regadenoson comprising a tonicity modifier.

### BACKGROUND OF THE INVENTION

Regadenoson is chemically described as 2-[4-[(methylamino) carbonyl]-1H-pyrazol-1-yl]-adenosine. Regadenoson is a selective A2A-adenosine receptor agonist that is a coronary vasodilator and has the following structure:

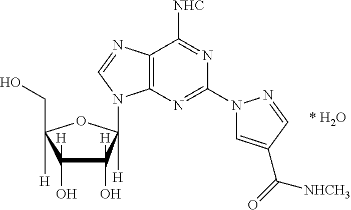

Regadenoson is used as a pharmacologic stress agent indicated for radionuclide myocardial perfusion imaging (MPI) in patients unable to undergo adequate exercise stress. Myocardial perfusion imaging (MPI) is a diagnostic technique useful for the detection and characterization of coronary artery disease. Perfusion imaging uses materials such as radionuclides to identify areas of insufficient blood flow. In Myocardial perfusion imaging (MPI), blood flow is measured at rest, and the result compared with the blood flow measured during exercise on a treadmill (cardiac stress testing), such exertion being necessary to stimulate blood flow. Unfortunately, many patients are unable to exercise at levels necessary to provide sufficient blood flow, due to medical conditions. Therefore, a pharmacological agent that increases cardiac blood flow for a short period of time would be of great benefit, particularly one that did not cause peripheral vasodilation.

Vasodilators such as dipyridamole have been used which is given as an infusion rather than a bolus and also it is non-selective for adenosine receptors and requires weight-based dosing. Adenoscar® (Adenosine) has been marketed as an adjuvant in perfusion studies using radioactive thalium-201, however its use is limited due to side effects, further the short half-life of adenosine necessitates continuous infusion during the procedure. Other potent and selective agonists for the A2A adenosine receptor are known such as MRE-0470 (Medco). Medco is used as an adjuvant in imaging. In general such compounds having high affinity towards A₂ adenosine receptor and consequently long duration of action is undesirable, which possibly prolong the duration of side-effects.

Regadenoson is a potent and useful selective A2A adenosine receptor agonist, has short duration of action and does not appear to require administration as a continuous infusion. Regadenoson and related compounds as well as methods for their manufacture and use in cardiac perfusion imaging are disclosed in U.S. Pat. Nos. 6,403,567, 6,642,210, 6,214, 807, 7655636, 8071566, 8106029 and 6,770,634, as well as published in U.S. patent application no. 2002-0012946 and Ser. No. 13/333,789.

Commercially, regadenoson is available as solution for intravenous injection under the brand name of Lexiscan® in the United States by Astellas. Lexiscan® is a sterile, clear and colorless nonpyrogenic solution for intravenous injection, available as a 1 mL in a 5-mL vial or pre-filed syringe. Each 1 mL in the 5-mL vial or prefilled syringe contains 0.084 mg of regadenosan monohydrate, corresponding to 0.08 mg regadenoson on an anhydrous basis, 10.9 mg dibasic sodium phosphate dihydrate or 8.7 mg dibasic sodium phosphate anhydrous, 5.4 mg monobasic sodium phosphate monohydrate, 150 mg propylene glycol, 1 mg edetate disodium dihydrate, and Water for Injection, with pH between 6.3 and 7.7.

U.S. Pat. No. 8,133,879 describes pharmaceutical compositions of regadenoson comprising of buffers specifically phosphate buffer, EDTA, carriers, cosolvents thereof intended for use as an intravenous injection.

U.S. Pat. No. 8,106,029 describes method of producing coronary vasodiation by administering pharmaceutical compositions of regadenoson as a single intravenous bolus dose.

U.S. application Ser. No. 13/333,789 describes a pharmaceutical composition including regadenoson—an adenosine A.sub.2A receptor agonist.

U.S. Pat. No. 7,655,636 describes method of producing coronary vasodilatation with little peripheral vasodilation comprising administering to a human a single dose of a pharmaceutical composition comprising regadenoson and at least one pharmaceutical excipient.

Regadenoson formulations have been taught to include chelating agents and phosphate buffer in the formulations to keep the formulation stable. European medicines agency document discloses that, possibly phosphorus from the phosphate buffer interacts with the glass vial surface and causes precipitation; thus a chelating agent is added to prevent catalytic hydrolysis of regadenoson caused by any trace metal ions and to prevent precipitation from a possible interaction of glass vial surface with phosphorus from the phosphate buffer. Regadenoson composition with a tonicity modifier were not disclosed in the prior art.

It is thus desired to develop a formulation that is devoid of any risk for leachables from primary packaging container and yet have a stable product. It has thus, surprisingly been found that formulations prepared without any phosphate buffer and chelating agents were found to be stable.

### SUMMARY OF THE INVENTION

Aspects of the present invention relates to pharmaceutical compositions of regadenoson including its pharmaceutically acceptable salts, solvates, hydrates and polymorphs thereof, in the form of solution for intravenous administration and preparations thereof.

Another aspect of the present invention is to provide the pharmaceutical composition comprising of regadenoson or its pharmaceutically acceptable salts, solvates, hydrates and polymorphs thereof, a tonicity modifier and other pharmaceutically acceptable adjuvants.

Another aspect of the present invention is to provide pharmaceutical compositions of regadenoson devoid of phosphate buffer.

Another aspect of the present invention is to provide pharmaceutical compositions of regadenoson devoid of chelating agents such as EDTA.

Another aspect of the present invention is to provide pharmaceutical compositions of regadenoson devoid of phosphate buffer and chelating agents such as EDTA.

Another aspect of the present invention is to provide pharmaceutical compositions of regadenoson devoid of chelating agent, phosphate buffer and contains a complexing agent.

Another aspect of the present invention is to provide pharmaceutical compositions of regadenoson devoid of phosphate buffer and contains a buffer other than a phosphate buffer.

Another aspect of the present invention provides manufacturing process for preparing solution compositions of regadenoson that does not comprise phosphate buffer.

Another aspect of the present invention provides manufacturing process for preparing solution compositions of regadenoson that does not comprise a chelating agent.

Another aspect of the present invention provides manufacturing process for preparing solution compositions of regadenoson that comprises of regadenoson, a tonicity modifier and other pharmaceutically acceptable adjuvants.

Another aspect of the present invention provides manufacturing process for preparing solution compositions of regadenoson that does not comprise of a chelating agent and phosphate buffer and may optionally contain a buffer other than a phosphate buffer.

Another aspect of the invention is to provide regadenoson solution composition comprising: a) regadenoson or a pharmaceutically acceptable salts, solvates and hydrates thereof; a tonicity modifier and pharmaceutically acceptable adjuvants thereof.

### DETAILED DESCRIPTION OF THE INVENTION

Aspects of the present invention relate to pharmaceutical formulations of regadenoson including its pharmaceutically acceptable salts, solvates or hydrates thereof, in the form of solution for intravenous administration and preparations thereof.

The formulations of the present invention are particularly suited for use in parenteral administration, more specifically by intravenous administration.

Another aspect of the present invention is to provide the pharmaceutical composition comprising of regadenoson or its pharmaceutically acceptable salts, solvates, hydrates and polymorphs thereof, a tonicity modifier and other suitable pharmaceutically acceptable adjuvants thereof.

Another aspect of the invention is to provide regadenoson solution formulations comprising: a) regadenoson or a pharmaceutically acceptable salts, solvates and hydrates thereof; and b) a pharmaceutically acceptable adjuvant, wherein the formulation does not contain a phosphate buffer and chelating agent and may optionally contain buffer other than phosphate buffer.

Another aspect of the invention is to provide regadenoson solution formulations comprising: a) regadenoson or a pharmaceutically acceptable salts, solvates and hydrates thereof, and b) a pharmaceutically acceptable adjuvant such as tonicity modifiers, cosolvent, stabilizers thereof, wherein the formulation does not contain a phosphate buffer and a chelating agent.

Another aspect of the invention is to provide regadenoson solution formulations comprising:

a) regadenoson or a pharmaceutically acceptable salts, solvates and hydrates thereof; and
b) a pharmaceutically acceptable adjuvant such as,

(i) Cosolvent

(ii) Tonicity modifier

(iii) Optionally a non-phosphate buffer

(iv) Optionally a complexing agent

(v) Optionally a stabilizer

The injectable formulation of the present invention comprises pharmaceutically acceptable adjuvants. The pharmaceutically acceptable adjuvant can be selected from tonicity modifiers, co-solvent, complexing agents, preservatives, anti-oxidants, stabilizers and any other suitable adjuvant thereof.

Tonicity modifiers are the compounds which make the composition isotonic with blood, may be added. Suitable tonicity modifiers include the following, but are not limited to sorbitol, dextrose, glycerol, mannitol, lactose, sucrose, sodium chloride, sodium bicarbonate, calcium chloride, potassium chloride, sodium lactate, Ringer's solution and lactated Ringer's solution, amino acids, trehalose and mixtures and salts thereof.

The tonicity modifiers used in the pharmaceutical composition is in the range of 0.05 to 30 percent; based on the total weight of the parenteral formulation.

The solubility of the drug can be increased by the addition of cosolvent in which the drug has good solubility. The use of cosolvents is an effective technique to enhance the solubility of the drug in the formulation. Suitable cosolvents include the following, but are not limited to N-methytpyrolidone (NMP), dimethylformamide (DMF), dimethyl sulfoxide (DMSO), N,N-dimethylacetamide (DMA), acetonitrile, M-PYROL, Ethanol, ethyl acetate, propylene glycol (PG), polyethylene glycol, glycerine, glycofurol, transcutol and mixtures thereof or their equivalents. Most preferred cosolvent is propylene glycol.

The cosolvent used in the pharmaceutical composition is in the range of 3 to 70 percent; based on the total weight of the parenteral formulation.

The pharmaceutical compositions of the present invention optionally contain one or more anti-oxidants and preservatives such as butylated hydroxyanisole (BHA), butylated hydroxyl toluene (BHT), glycine, citric acid, d,l-.alpha.-tocopherol, monothioglycerol, ascorbic acid, propyl gallate, aminoacids and mixtures thereof.

The pharmaceutical compositions of the present invention optionally contain stabilizers such as glycine, dextran, diethylene glycol monoethyl ether, tweens, transcutol, Cremophores, labrasal mono, di and tri glycerides, alkoxylated phenols, diphends, fatty acid esters of glycerol and sorbitol, ethoxylated ethers, ethoxylated esters, propylene carbonate, polysorbate, nitrilotriacetate; disodium nitrilotriacetate, phospholipids and mixtures thereof.

The pharmaceutical compositions of the present invention optionally contains buffers such as citrate buffer, glutamate, bicarbonate, tartrate, benzoate, lactate, gluconate, glycine, TRIS buffer, acetate buffer, boric add buffer or any other suitable buffer and mixtures thereof, but not selected from phosphates buffer. Compositions of the present invention will have a pH from 4 to 10.

The pharmaceutical composition of the present invention optionally contains complexing agents such as cyclodextrins. Cyclodextrins are a series of natural cyclic oligosaccharides composed of six, seven, eight, or more D (+) glycopyranose units linked by alpha 1, 4 linkages. Suitable cyclodextrins include .alpha.-cyclodextrin, .beta.-cyclodextrin, .gamma.cyclodextrin and derivatives thereof, including hydrophobic derivatives, hydrophilic derivatives, charged cyclodextrins, and the like.

The pharmaceutical compositions of the present invention optionally contain an aqueous vehicle such as water. A composition prepared by using aqueous vehicle, water is added in sufficient amounts to bring the mixture to sufficient volume. In case of non-aqueous compositions, solvent is added in sufficient amounts to bring the mixture to sufficient volume.

The compositions of the present invention comprises: a) regadenoson or a pharmaceutically acceptable salts, solvates, hydrates thereof; and b) a pharmaceutically acceptable adjuvant such as co-solvent and a tonicity modifier wherein the composition has a pH from about 4 to 10.

The compositions of the present invention comprise solutions which are ready to use in which regadenoson is present in a suitable concentration for direct administration without any dilution. The compositions of the present invention can be prepared by the following process; Dissolve the required excipients in water then add regadenoson or its salts or hydrates thereof. Make up the final volume to q.s with water for injection or any suitable solvent. Fill in to a suitable container. Suitable sterilization techniques are used to keep the composition sterile.

The composition of the present invention could be packed in vials or pre filed syringes. Prefilled medical devices are medical devices that are filled by the manufacturer at the time of assembly and are shipped in a ready-to-use condition to the healthcare provider. Prefilled medical devices have the advantage of convenience and ease of application with reduced risk of contamination of the contents of the device, such as a drug solution. Vials and Profiled syringes can be made of or lined with Polypropylene, polycarbonate, cyclic olefins such as cyclic olefin copolymers (COCs) and cyclic olefin polymers (COPs) crystal Zenith® (CZ), plastic, glass or any suitable material thereof. The cyclic olefins have very attractive properties for the manufacture of pre-fillable syringes and vials such as excellent optical transmission, low birefringence, high purity, low moisture uptake and an excellent moisture barrier. Most preferred material would be CZ, cyclic olefin copolymers (COCs) and cyclic olefin polymer (COPs). The stopper assembly may be optionally laminated or coated with an inert material, selected from the group consisting of tetrafluoroethylene resin, an ethylenetetrafluoroethylene resin, a UHMW polyethylene resin, or various carbon coatings thereof.

The compositions prepared according to the present invention could be sterilized using aseptic filtration or terminal sterilization or both. Sterilization is desired so as to reduce or to eliminate the risk of exposure to potential pathogens contained within the device. Terminal sterilization is defined as the sterilization of the finished pharmaceutical product while in the final container for delivery. Terminal sterilization can be achieved by the following techniques, such as γ-irradiation, e-Beam, microwave, moist heat sterilization or any suitable sterilization techniques thereof.

The following examples further describe certain specific aspects and embodiments of the present invention and demonstrate the practice and advantages thereof. It is to be understood that the examples are given by way of illustration only and are not intended to limit the scope of the invention in any manner.

### Example 1

### Composition

| S. No | Ingredients | Qty/mL |
|---|---|---|
| 1. | Regadenoson | 0.08 mg |
| 2. | Sodium chloride | 6.6 mg |
| 3. | Propylene Glycol | 150 mg |
| 4. | Water for Injection | QS to 1 mL |

Manufacturing procedure:
1) Take required quantity of propylene glycol in a SS jacketed manufacturing vessel and add required quantity of water and stir well to get a uniform solution.
2) Add required quantity of Regadenoson to the above solution and stir well until it dissolves.
3) Dissolve sodium chloride in water and add this solution to the above solution.
4) Make up the final volume up to q.s by water for Injection and stir properly to get a homogeneous solution.
5) Filter the solution through 0.22 micron filter.
6) Fill desired volume of bulk solution into a PFS or vials and dose with stopper.

The product is stored for various time periods at various conditions and samples were analyzed for pH of the solution, for drug content and impurities. Stability studies were performed in both CZ and glass vials. Even the stability studies were performed in pre filled syringes also. Results are tabulated in Tables 1, 2 and 3.

TABLE 1
Stability data for the product obtained from Example 1; wherein
the sample is packed in 5 mL USP Type I glass vial.

| | | Parameters | | | | | |
|---|---|---|---|---|---|---|---|
| | | | | | | Related Substances | |
| | | | | | | Unknown | % Total |
| | Descrip- | Identifi- | pH of the | Osmo- | Assay | Impurity | impuri- |
| Interval | tion | cation | Solution | larity | (%) | (RRT 0.70) | ties |

25° C. ± 60% RH

| | | | | | | | |
|---|---|---|---|---|---|---|---|
| Initial | C | C | 7.19 | 101 | 97.9 | 0.18 | 0.36 |
| 1 Month | C | C | 7.08 | 102 | 99.8 | 0.21 | 0.59 |
| 2 Months | C | C | 7.28 | 101 | 99.9 | 0.21 | 0.63 |
| 3 Months | C | C | 7.23 | 104 | 99.7 | 0.22 | 0.69 |

40° C. ± 75% RH

| | | | | | | | |
|---|---|---|---|---|---|---|---|
| Initial | C | C | 7.19 | 101 | 97.9 | 0.18 | 0.36 |
| 1 Month | C | C | 7.13 | 102 | 100.2 | 0.23 | 0.61 |
| 2 Months | C | C | 7.13 | 101 | 99.8 | 0.28 | 0.72 |
| 3 Months | C | C | 7.09 | 102 | 100.2 | 0.29 | 0.78 |

60° C.

| | | | | | | | |
|---|---|---|---|---|---|---|---|
| Initial | C | C | 7.19 | 101 | 97.9 | 0.18 | 0.36 |
| 1 Week | C | C | 6.81 | 98 | 100.4 | 0.41 | 0.97 |
| 2 Week | C | C | 7.14 | 91 | 96.8 | 0.41 | 0.99 |
| 1 Month | C | C | 7.17 | 121 | 100.5 | 0.61 | 1.63 |

*C: Complies

TABLE 2
Stability data for the product obtained from Example
1; wherein the sample is packed in 5 mL CZ vial.

| | Parameters | | | | | | |
|---|---|---|---|---|---|---|---|
| | | | | | | Related Substances | |
| | | | | | | Unknown | % Total |
| | Descrip- | Identifi- | pH of the | Osmo- | Assay | Impurity | impuri- |
| Interval | tion | cation | Solution | larity | (%) | (RRT 0.70) | ties |

25° C. ± 60% RH

| | | | | | | | |
|---|---|---|---|---|---|---|---|
| Initial | C | C | 7.05 | 115 | 101.0 | 0.06 | 0.41 |
| 1 Month | C | C | 7.04 | 102 | 100.8 | 0.10 | 0.44 |
| 2 Months | C | C | 7.02 | 101 | 100.2 | 0.09 | 0.45 |
| 3 Months | C | C | 7.01 | 100 | 99.5 | 0.10 | 0.55 |

40° C. ± 75% RH

| | | | | | | | |
|---|---|---|---|---|---|---|---|
| Initial | C | C | 7.05 | 115 | 101.0 | 0.06 | 0.41 |
| 1 Month | C | C | 7.11 | 101 | 101.5 | 0.10 | 0.43 |
| 2 Months | C | C | 7.02 | 105 | 100.1 | 0.09 | 0.44 |
| 3 Months | C | C | 7.01 | 104 | 99.6 | 0.10 | 0.51 |

60° C.

| | | | | | | | |
|---|---|---|---|---|---|---|---|
| Initial | C | C | 7.05 | 115 | 101.0 | 0.06 | 0.41 |
| 1 Week | C | C | 6.25 | 98 | 101.2 | 0.08 | 0.50 |
| 2 Week | C | C | 7.09 | 95 | 101.0 | 0.09 | 0.55 |
| 1 Month | C | C | 7.10 | 118 | 101.5 | 0.08 | 0.57 |

*C: Complies

TABLE 3
Stability data for the product obtained from Example 1; wherein the sample is packed in PFS.

| | Parameters | | | | | | | |
|---|---|---|---|---|---|---|---|---|
| | | | | | Related Substances | | | |
| | | | | | Unknown | % Total | Low wave | Extractables |
| | Descrip- | Identifi- | pH of the | Assay | Impurity | impuri- | length | and |
| Interval | tion | cation | Solution | (%) | (RRT 0.70) | ties | analysis | leachables |

Storage Conditions: 40° C. ± 75% RH
5 ml CZ Syringe with PTFE laminated stopper and cap.

| | | | | | | | | |
|---|---|---|---|---|---|---|---|---|
| Initial | C | C | 6.76 | 99.4 | 0.10 | 0.16 | No extra | No extra |
| | | | | | | | peaks were | peaks were |
| | | | | | | | found | found |
| 1 Month | C | C | 6.73 | 100.5 | 0.07 | 0.20 | No extra | No extra |
| | | | | | | | peaks were | peaks were |
| | | | | | | | found | found |
| 2 Months | C | C | 6.72 | 100.7 | 0.10 | 0.29 | No extra | No extra |
| | | | | | | | peaks were | peaks were |
| | | | | | | | found | found |
| 3 Months | C | C | 6.81 | 100.5 | 0.10 | 0.39 | No extra | No extra |
| | | | | | | | peaks were | peaks were |
| | | | | | | | found | found |

5 ml COC Syringe with kokoku rubber stopper and leur lock fitting

| | | | | | | | | |
|---|---|---|---|---|---|---|---|---|
| Initial | C | C | 6.76 | 99.4 | 0.10 | 0.16 | No extra | No extra |
| | | | | | | | peaks were | peaks were |
| | | | | | | | found | found |
| 1 Month | C | C | 6.78 | 100.1 | 0.07 | 0.21 | No extra | No extra |
| | | | | | | | peaks were | peaks were |
| | | | | | | | found | found |
| 2 Months | C | C | 6.79 | 99.7 | 0.11 | 0.26 | No extra | No extra |
| | | | | | | | peaks were | peaks were |
| | | | | | | | found | found |
| 3 Months | C | C | 6.95 | 100.6 | 0.10 | 0.33 | No extra | No extra |
| | | | | | | | peaks were | peaks were |
| | | | | | | | found | found |

*C: Complies

From the above results it is evident that the formulations devoid of chelating agent such as EDTA and phosphate buffer, were found to be stable. There was no leaching observed. Formulations containing a tonicity modifier exhibited improved stability which was surprising.

### Example 2

### Comparative Example—Compositions Containing EDTA and without EDTA

| | | Qty. required | Qty. required | Qty. required |
|---|---|---|---|---|
| | | (With | (Without | (With |
| S. | Name of | EDTA) | EDTA) | EDTA) |
| No | Ingredient | Qty per vial | Qty per vial | Qty per vial |
| 1 | *Regadenoson | 0.4 mg | 0.4 mg | 0.4 mg |
| | monohydrate | | | |
| 2 | Dibasic sodium | — | — | 43.5 mg |
| | phosphate anhydrous | | | |
| 3 | Monobasic | — | — | 27 mg |
| | sodium phosphate | | | |
| | monohydrate | | | |
| 5 | Sodium chloride | 33 mg | 33 mg | — |
| 6 | Propylene glycol | 750 mg | 750 mg | 750 mg |
| 7 | EDTA | 5 mg | — | 5 mg |
| 8 | Water | Q.S to 5 ml | Q.S to 5 ml | Q.S to 5 ml |

Samples obtained from the example 2 were analyzed for total impurities, pH and osmolarity. Results are tabulated in Table 4.

TABLE 4

| | | Formulations with EDTA | | | | Formulations without EDTA | |
|---|---|---|---|---|---|---|---|
| | | (45 min Autoclaving) | | | | (45 min Autoclaving) | |
| | Without | Sodium chloride | | Phosphate buffer | | Sodium chloride | |
| | Autoclaving | Glass | CZ | Glass | CZ | Glass | CZ |
| Total | 0.14 | 0.92 | 1.10 | 0.38 | 0.39 | 0.29 | 0.17 |
| impurities | | | | | | | |
| pH | 7.06 | 4.39 | 4.17 | 7.03 | 7.02 | 6.71 | 6.06 |
| Osmolarity | 2242 | 2083 | 2180 | 1881 | 1842 | 2114 | 2012 |

From the above results it is evident that compositions containing EDTA and sodium chloride the impurities content are relatively high, having undesirable pH.

It is surprisingly found that the compositions comprising sodium chloride devoid of phosphate buffer and EDTA were found to be stable.

### Example 3

### Composition

| | | |
|---|---|---|
| S. No | Ingredients | Qty/mL |

| | | | |
|---|---|---|---|
| 1. | Regadenoson | 0.08 | mg |
| 2. | Dimethyl acetamide | 0.2 | mL |

| | | |
|---|---|---|
| 3. | Water for Injection | QS to 1 mL |

Manufacturing Procedure:
1) Take required quantity of Dimethyl acetamide (DMA) in a SS vessel and add Regadenoson to the dimethyl acetamide (DMA) and stir well until it dissolves completely.
2) Make up the final volume q.s by water and stir properly to get a uniform solution.
3) Filter the solution through 0.22 micron filter.
4) Fill desired volume of bulk solution into a PFS or vials and close with stopper.

### Example 4

### Composition

| | | |
|---|---|---|
| S. No | Ingredients | Qty/mL |

| | | | |
|---|---|---|---|
| 1. | Regadenoson | 0.08 | mg |
| 2. | Dimethyl Acetamide | 0.2 | mL |
| 3. | Propylene glycol | 150 | mg |

| | | |
|---|---|---|
| 4. | Water for Injection | QS to 1 mL |

Manufacturing Procedure:
1) Take required quantity of Dimethyl Acetamide (DMA) in a SS vessel to which add sufficient amount of Regadenoson and stir well until it dissolves completely.
2) Add required amount of propylene glycol to the Dimethyl Acetamide (DMA) solution.
3) Make up the final volume to q.s by water and stir properly to get a uniform solution.
4) Filter the solution through 0.22 micron filter.
5) Fill the desired volume of bulk solution into a PFS or vials and dose with stopper.

### Example 5

### Composition

| S. No | Ingredients | Qty/mL |
|---|---|---|
| 1. | Regadenoson | 0.08 mg |
| 2. | Propylene glycol | 150 mg |
| 3. | Water for Injection | QS to 1 mL |

Manufacturing Procedure:
1) Take required quantity of propylene glycol in a SS vessel containing water and stir well.
2) Add Regadenoson to the propylene glycol solution and stir well until the drug is completely dissolved.
3) Make up the final volume to q.s by water and stir properly to get a uniform solution and filter through 0.22 micron filter.
4) Fill the desired volume of bulk solution into PFS or vials and close with stopper.

### Example 6

### Composition

| S. No | Ingredients | Qty/mL |
|---|---|---|
| 1 | Regadenoson | 0.08 mg |
| 2 | Tris buffer | 1 mg |
| 3 | Propylene glycol | 150 mg |
| 4 | Water for Injection | QS to 1 mL |

Manufacturing Procedure:
1) Take required quantity of propylene glycol in a SS vessel containing water and stir well.
2) Add Regadenoson to the propylene glycol solution and stir well, until the drug is completely dissolved.
3) Add Tris buffer for pH adjustment and stir well to get a uniform solution.
4) Make up the final volume to q.s by water and stir properly to get a uniform solution and filter through 0.22 micron filter.
5) Fill desired volume of bulk solution into PFS or vials and close with stopper.

### Example 7

### Composition

| S. No | Ingredients | Qty/0.5 mL |
|---|---|---|
| 1 | Regadenoson | 0.08 mg |
| 2 | Dimethylacetamide | QS to 0.5 mL |

Manufacturing procedure:
1) Take required quantity of DMA (80%) in a 88 vessel and add Regadenoson; stir properly, until the drug is completely dissolved.
2) Make up the final volume to q.s by DMA and stir properly to get a uniform solution.
3) Filter the solution through 0.22 micron filter.
4) Fill desired volume of solution into PFS or vials and close with stopper.

1-21. (canceled)

22. A parenteral pharmaceutical formulation comprising regadenoson, a tonicity modifier, water and pharmaceutically acceptable adjuvants.

23. The parenteral pharmaceutical formulation of regadenoson of claim 22, wherein the amount of tonicity modifier is from 0.05 to 30 percent; based on the total weight of the parenteral formulation.

24. The formulation of claim 23, wherein the tonicity modifier is selected from the group comprising sorbitol, dextrose, glycerol, mannitol, lactose, sucrose, sodium chloride, sodium bicarbonate, calcium chloride, potassium chloride, sodium lactate, Ringer's solution and lactated Ringer's solution, amino acids, trehalose and mixtures thereof.

25. The formulation of claim 23 wherein the tonicity modifier is sodium chloride.

26. The formulation of claim 25 which further comprises a cosolvent.

27. The formulation of claim 26, wherein the cosolvent is selected from the group comprising N-methylpyrrolidone (NMP), dimethylformamide (DMF), dimethyl sulfoxide (DMSO), N,N-dimethylacetamide (DMA), acetonitrile, M-PYROL, Ethanol, ethyl acetate, propylene glycol (PG), polyethylene glycol, glycerine, glycofurol, transcutol and mixtures thereof.

28. The formulation of claim 26, wherein the cosolvent is propyleneglycol.

29. A parenteral pharmaceutical formulation comprising regadenoson, sodium chloride, propylene glycol and water.

30. A parenteral pharmaceutical formulation of regadenoson that is free of chelating agents and/or phosphate buffer.

31. The formulation of claim 30 comprising regadenoson, sodium chloride, propylene glycol and water.
